(19) United States
(12) Patent Application Publication
(10) Pub. No.: US 2017/0196910 A1
(43) Pub. Date: Jul. 13, 2017

(54) GAMMA DELTA T CELLS AND USES THEREOF

(71) Applicant: TC Biopharm LTD, Lothian (GB)

(72) Inventors: Michael LEEK, Edinburgh, Lothian (GB); Adele HANNIGAN, Glasgow, Strathclyde (GB)

(73) Assignees: TC Biopharm LTD, Lothian (GB); TC Biopharm LTD, Lothian (GB)

(21) Appl. No.: 15/325,012

(22) PCT Filed: Jul. 8, 2015
(86) PCT No.: PCT/GB2015/051985

§ 371 Date: Jan. 9, 2017

(30) Foreign Application Priority Data
Jul. 9, 2014 (GB) 1412175.0
Aug. 29, 2014 (GB) 1415379.5
Apr. 15, 2015 (GB) 1506423.1

(51) Int. Cl. A61K 35/17; C12N 5/0783; A61K 45/06

(52) U.S. Cl. CPC A61K 35/17; A61K 45/06; C12N 5/0638; C12N 2501/2302; C12N 2501/999

(57) ABSTRACT

A method of preparing and using gamma delta T cells in the allogeneic or autologous treatment of subjects suffering from virus infection, fungal infection, protozoal infection and cancer.

### FIELD OF THE INVENTION

This application relates to methods of preparing and using gamma delta T cells and in particular, the use of gamma delta T cells in allogeneic or autologous recipient subjects for the treatment of conditions including virus infection, fungal infection, protozoal infection and cancer.

### BACKGROUND

Allogeneic stem cell transplantation (allo-SCT) has been suggested and trialled in relation to hematologic malignancies. However, a major disadvantage of such allogeneic therapy is the high incidence of graft failure and graft versus host disease (GVHD). HLA-haplo identical donors have been utilised to try to improve the outcome of such transplantations. Additionally, T cell depleted HLA-matched-SCT has been attempted, using ex vivo depletion of graft T cells to reduce GVHD; however, it is considered that this leads to an increased risk of graft failure.

If the recipient is intensively conditioned to reduce the risk of graft failure and receives a T cell depleted graft, it is considered that immune reconstitution is unacceptable and too many patients would die from opportunistic infections.

Gamma delta T lymphocytes represent a minor subset of cells within peripheral blood in humans (less than 10%). Gamma delta T cells expressing Vγ9Vδ2 (gamma 9 delta 2) T cell receptor recognise the endogenous isopentenyl pyrophosphate (IPP) that is over produced in cancer cells as the result of a dysregulated mevalonate pathway. The ability of gamma delta T lymphocytes to produce abundant pro inflammatory cytokines like IFN-gamma, their potent cytotoxic effective function and MHC-independent recognition of antigens makes them an important layer of cancer immunotherapy. Gamma delta T cells have been indicated to be able to kill many different types of tumour cell lines and tumours in vitro, including leukaemia, neuroblastoma and various carcinomas. Further, it has been demonstrated that gamma delta T cells can recognise and kill many different differentiated tumour cells either spontaneously or after treatment with different bisphosphonates, including zoledronate. Human tumour cells can efficiently present pyrophosphomonoester compounds to gamma delta T cells inducing their proliferation and IFN-gamma production.

Presently, two strategies have been used with gamma delta T cell tumour immune- therapy. A first method involves the adoptive cell transfer of in vitro expanded gamma delta T cells back to a patient (i.e. an autologous treatment). The second method involves in vivo therapeutic application of gamma delta T cell stimulating phosphoantigens or amino bisphosphonates together with low dose recombinant IL-2.

Autologous transplantation strategies of gamma delta T cells have been utilised to overcome the disadvantages noted above for allogeneic stem cell transplantation. As part of such autologous transplantation techniques, methods of inducing and culturing sufficient numbers of gamma delta T cells for exerting therapeutic effect autologously have been previously disclosed, for example US 2002/0107392. However, autologous treatment strategies suffer from a number of disadvantages.

Thus, alternative and/or improved autologous and allogeneic treatment strategies are required.

### SUMMARY OF THE INVENTION

Whilst gamma delta T cell therapy in relation to cancer therapy has been discussed in relation to autologous use, it has to date not been considered to provide such gamma delta T cell therapy allogeneically. It is considered that such allogeneic use of gamma delta T cell therapy has not been considered typically due to potential problems linked to immune-system mediated rejection. The inventors surprisingly consider that gamma delta T cells do not typically cause graft versus host disease, and that the selection of gamma delta T cells for allogeneic transplantation could allow T cells to be provided to a recipient with a minimal risk of graft versus host disease. Gamma delta T cells are not MHC restricted (Tanaka Y et al., 1995). The inventors consider that this will allow gamma delta T cells to be used in allogeneic transplantation to provide a viable therapy wherein gamma delta T cells are capable of targeting cells for cytolysis independently of MHC-haplotype. In view of the lack of recognition of MHC-presented antigens by gamma delta T cells, the present inventors consider that the risk of GVHD would be minimised in a high purity allogeneic transfer of gamma delta T cells sufficiently purified from other leukocytes including B cells and alpha beta T cell receptor (TCR) T cells. Additionally, it is considered there will be a low chance of graft rejection due to the immuno-compromised state of the recipient in certain disease states, including but not limited to patients with severe viral infections for example Ebola , HIV and Influenza as well as PTLD-EBV patients and those with other cancer types.

As noted, previous treatment strategies have included T cell removal from donor blood, in particular peripheral blood, using a negative selection or positive selection methodology, prior to allogeneic stem cell transplantation.

The present inventors have determined a method to allow collection of cells from a donor subject and processing of such donor cells to allow the provision of sufficient numbers of gamma delta T cells allogeneically to a recipient subject, such that the gamma delta T cells can exert a therapeutic effect to the recipient subject.

By way of example, the inventor's gamma delta T cell expansion method may comprise the isolation of peripheral blood mononuclear cells (PBMCs) from blood or leukapheresis material using density gradient centrifugation. Isolated PBMCs may be cryopreserved prior to expansion in culture, whilst plasma is co-extracted and retained as an autologous excipient for use in subsequent gamma delta T cell culturing steps. In embodiments freshly isolated PBMCs (or those resuscitated from cryopreservation) are inoculated into growth media containing human recombinant IL-2 (e.g. at a concentration of up to 1000 U/ml) and Zoledronic acid (e.g. 5 μM). The yδ T lymphocyte population may be activated and selectively proliferated from the PBMCs via the addition of zoledronic acid (day 0) and the continuous inclusion of IL-2 over a 14 day culture period. The cell suspension may be serially expanded (typically at a 1:2 split ratio) over this time period. 14 days after culture initiation the cells can be harvested and resuspended in lactated ringers solution and HSA prior to transfer to an infusion bottle containing 100 ml saline solution.

Following expansion, in embodiments, the gamma delta T cell product meets the following minimum specifications; greater than 80% of total cells are T lymphocytes (CD3 positive), gamma delta T lymphocytes comprise 60% or greater of the total T lymphocyte population (Vgamma9 positive), NK cells are less than 25% of the total T lymphocyte population (CD3 negative/CD56 positive), Cytotoxic T cells are below 10% of total T lymphocyte population (CD3/CD8 positive) and T helper cells are below 5% of total T lymphocyte population (CD3/CD4 positive). In embodiments, cell populations meeting these specifications can be used as the starting material for the generation of high purity allogeneic cell banks which will aim to have greater than 99% gamma delta T cells.

According to a first aspect of the present invention there is provided a process for providing gamma delta T cells allogeneically to a second subject comprising the steps
providing a sample comprising gamma delta T cells from a first subject;
culturing the gamma delta T cells to allow them to be administered to a second subject.

In embodiments, the step of providing can include a step of collecting the gamma delta T cells from a first subject. The collection can be from a donor subject wherein the donor subject has no immediate perceived health conditions or from umbilical cord blood material. Suitably the recipient subject may be a vertebrate, for example a mammal, for example a human, or commercially valuable livestock, a research animal, a horse, cow, goat, rat, mouse, rabbit, pig, and the like. In embodiments the first and second subjects can be human. As will be understood, in the context of the present invention, the first subject is a donor subject from whom gamma delta T cells are collected, and the cells are used in the allogeneic treatment of a different second (recipient) subject. Suitably, the first subject has a pre-disease state. The term “pre-disease” state as used herein covers the absolute term of “healthy”, “no disease”, “and the relative term of a graduation in a disease potential progression”, “healthier than” or “less diseased than” a post diseased state. Since “pre-disease” can be defined by a time prior to the first subject being diagnosed with a disease, the first subject can be healthy in an absolute term or might already have the disease where the disease is not yet manifested itself or been diagnosed or detected. In embodiments the first aspect of the invention comprises the step of culturing gamma delta T cells obtained from a first subject to allow the gamma delta T cells to be provided to a second subject.

In embodiments the gamma delta T cells can be collected from peripheral blood or peripheral blood mononuclear cells obtained following apheresis or leukapheresis or from umbilical cord blood. Ex vivo expansion of gamma delta T cells from peripheral blood will preferentially give rise to gamma delta T cells of the Vγ9Vδ2 phenotype when activated with phosphoantigens or aminobisphosphonates. The use of umbilical cord blood as starting material for ex vivo expansion permits the selective expansion of several T cell receptor (TCR) subtypes dependent upon the activating antigen. These TCR isotypes may include may include any gamma delta TCR pairing from Vγ1-9 and Vδ1-8, for example, but not limited to Vδ2, Vδ2 and Vδ3 TCR variants. Gamma delta T cells of discrete subtypes recognise distinct antigens and would therefore exhibit differing levels of cytotoxicity dependent upon the antigens presented by the target cells. The relative abundances of each delta TCR subtype is dependent largely upon the culturing conditions and specific antigens presented. Culturing conditions may be tailored to preferentially expand a desired TCR isotype from umbilical cord blood. For example, gamma delta T cells expressing a singular TCR isotype may be more efficacious in the treatment of a particular cancer type or for the treatment of a specific viral infection.

In embodiments the collecting step can comprise the step of administering to the first subject a gamma delta T cell potentiating agent, prior to collecting the gamma delta T cells from the first subject.

In embodiments the method of collecting the gamma delta T cells can comprise the step of administering to the first subject a potentiating agent such as a growth factor which induces white cell mobilization from the bone marrow such as G-CSF, an aminobisphosphonate, in particular pamidronic acid, alendronic acid, zoledronic acid, risedronic acid, ibandronic acid, incadronic acid, a salt therefor and/or a hydrate thereof, TNFalpha or interleukin 2 (Meraviglia S et al., 2010)

In an embodiment the process can comprise any one or more of the steps of:—
providing blood, for example umbilical cord blood or apheresis/leukophoresis derived cells from a first subject (donor),
separating peripheral blood mononuclear cells (PBMCs) or cord blood mononuclear cells (CBMC) from the blood,
adding amino bisphosphonate and a target antigen to the PBMCs or CBMCs, and
culturing the PBMCs or CBMCs to proliferate/induce target antigen specific cytotoxic T cells (CTLs) and gamma delta T cells and optionally
co-culturing the PBMCs or CBMCs or T cells with artificial antigen presenting cells (aAPC) to proliferate/induce target antigen specific cytotoxic T cells (CTLs) and gamma delta T cells.

The present inventors consider that providing gamma delta T cells that are substantially isolated from other components of whole blood will reduce the graft failure when those substantially isolated gamma delta T cells are allogeneically administered to a second subject. The process to provide gamma delta T cells allogeneically may include a step of active purification for example isolating gamma delta T cells from a mixed cell population using anti-gamma delta T cell receptor antibodies. Consequently, the process of the present invention may include a step of purifying gamma delta T cells from whole blood, or components thereof. As less than 10% of peripheral blood by total number of cells is composed of gamma delta T cells, purifying a sample of whole blood, or components thereof, so that more than 10% by mass of the sample consists of gamma delta T cells is considered to enhance the effectiveness of allogeneically treating the recipient subject. Consequently, the process for the present invention may include the step of purifying or expanding a sample of whole blood, or components thereof, in order to achieve a greater than 10, 25, 50, 75, 85, 90, 95 or 98% of the total number of cells in the purified sample being gamma delta T cells. It is considered that purifying or expanding a sample of whole blood or components thereof to achieve a greater than 10, 25, 50, 75, 85, 90, 95 or 98% of the total number of cells in the purified sample being gamma delta T cells whilst reducing cells in the sample which would lead to immune response and/or graft failure will allow allogeneic transfer of gamma delta T cells.

Any method known to the skilled person that is capable of purifying gamma delta

T cells from whole blood, umbilical cord blood or components thereof, can form part of the present invention. Clearly, the purification step should not affect or minimally affect the viability of the gamma delta T cells. For example, the following steps may be used in combination, or alone, to achieve the aforementioned purification of the gamma delta T cells: a process of dialysis (e.g. apheresis and/or leukophoresis); differential centrifugation; growth of gamma delta T cells in culture (e.g. preferential growth in culture).

The step of purification can, at least in part, be carried out during the culturing step. For example, during the culturing step, addition of at least one or a combination of specific components such as aminobisphosphonate in particular pamidronic acid, alendronic acid, zoledronic acid, risedronic acid, ibandronic acid, incadronic acid, a salt therefor and/or a hydrate thereof allows the gamma delta T cells to be selectively expanded in a culture. Purification during cell culture may also be achieved by the addition of synthetic antigens such as phosphostim/bromohalohydrin pyrophosphate (BrHPP), synthetic isopentenyl pyrophosphate (IPP), (E)-4-Hydroxy-3-methyl-but-2-enyl pyrophosphate (HMB-PP) or co-culture with artificial antigen presenting cells (aAPC) (Wang et al., 2011). The addition of such components provides a culturing environment which allows for positive selection of gamma delta T cells typically at 70% or greater by number of total cells in the purified sample.

An aminobisphosphonate can be added any time from the first day of culturing the gamma delta T cells. An aminobisphosphonate can be added at a concentration of 0.05 to 100 micromolar, preferably 0.1 to 30 micromolar to the peripheral blood mononuclear cells. Suitably, the bisphosphonate is an analogue of pyrophosphoric acid and is a compound in which the O (oxygen atom) of the pyrophosphoric acid skeleton P-O-P is substituted with C (carbon atom) (P-C-P).

It is generally used as a therapeutic drug for osteoporosis. The aminobisphosphonate refers to a compound having N (nitrogen atom) among the bisphosphonates. For example, the aminobisphosphonate used in the present invention is not particularly limited; aminobisphosphonates and the like as disclosed in WO 2006/006720 and WO 2007/029689 may be used. Specific examples thereof include pamidronic acid, its salt and/or their hydrate, alendronic acid, its salt and/or their hydrate, and zoledronic acid, its salt and/or their hydrate (Thompson K. et al., 2010). The concentration of the aminobisphosphonates is preferably 1 to 30 μM for pamidronic acid, its salt and/or their hydrate, 1 to 30 μM for alendronic acid, its salt and/or their hydrate, and 0.1 to 10 μM for zoledronic acid, its salt and/or their hydrate. Here, 5 μM zoledronic acid is added as an example.

Suitably, when the culture period is 7 days or more, a cell group comprising gamma delta T cells may be obtained with high purity; however, the culture is preferably performed for about 14 days to further increase the number of gamma delta T cells.

In embodiments, the period of culturing may be about 7 days or more. Suitably the period of culturing may be performed for about 14 days or greater to achieve high numbers of substantially purified gamma delta T cell populations

Culturing is typically performed for 14 days, after which time gamma delta T cells cease to continue exponential proliferation. However, certain embodiments provide for the extended culture and selective expansion of gamma delta T cells to greater numbers. Such embodiments include the provision of synthetic antigens to the culture (e.g. synthetic IPP, DMAPP, Br-HPP, HMB-PP), cyclic exposure to artificial or irradiated antigen presenting cells, the provision of immobilised antigens or antibodies or the use of umbilical cord blood as a starting material for cell culture.

Suitably, cells may be cultured in this environment for a period of at least 7 days to reset their cell surface receptor profile following a minimum of at least two population doublings.

Optionally, the step of culturing the gamma delta T cells may include steps for changing the gamma delta T cell surface receptor profile (Iwasaki M. et al., 2011).

For example, the culture step may involve one or more sub-steps that reduce or eliminate one or more gamma delta T cell surface receptor type present in gamma delta T cells provided in the sample from the first subject. Such steps may be seen to “reset” or “partially reset” the receptor profile of the gamma delta T cells back to a naïve or partially naïve form. It is contemplated that such resetting enhances the gamma delta T cells' ability to treat cancer and viral infection. It is known that some T cell receptors can be induced by the presence of cancer or viruses in the subject from which the T cells are derived, and it has been found that these receptors can in some cases inhibit the responsiveness to tumour or viral infection by the T cells. Consequently, removing such receptors may increase the efficaciousness of the gamma delta T cells of the present invention.

The reduction or elimination of one or more gamma delta T cell receptor type may be achieved by the process of the present invention by culturing the gamma delta T cells derived from the first subject over a number of days in which the cell population is increased in size a number of times. For example, cells may be cultured for a period of at least 7 days to reset their cell surface receptor profile following a minimum of at least two population doublings.

In cases where the gamma delta T cell surface receptor profile has been reset, cell surface receptors including for example immune checkpoint inhibitors which were present on primary, uncultured gamma delta cells such as tumour-specific cell surface receptors B7-H1/PD-L1, B7-DC/PD-L2, PD-1 and CTLA-4 may be rendered absent or substantially reduced in number during the culture expansion period.

The culturing step may further include a step of monitoring the surface receptor profile of the gamma delta T cells in order to determine the appropriate duration of the culturing step required in order to significantly decrease or remove selected gamma delta T cell surface receptors (for example, any one or any combination of the receptors discussed above (B7-H1/PD-L1, B7-DC/PD-L2, PD-1 and CTLA-4). The process of monitoring gamma delta T cell receptors may, for example, be carried out using flow cytometry techniques, such as those outlined by Chan D. et. al., 2014. Briefly, antibodies specific for immune checkpoint inhibitor receptors and/or ligands will be used to identify sub-populations of gamma delta T cells (co-stained with anti-Vgamma9 for example) expressing immune checkpoint inhibitors on their cell surface.

Additionally, or optionally, the culturing step of the present invention may include step(s) that induce(s) the expression in the gamma delta T cells of gamma delta

T cell surface receptor types that were not present on the surface of the uncultured gamma delta cells when extracted from the first subject, or a step(s) that induce an increase in the amount of expression of cell surface receptor type(s) that were present on the surface of the uncultured gamma delta cells when extracted from the first subject. This may be achieved by challenging the gamma delta T cells with an antigen derived from a cancer, bacterium, fungi, protozoa or a virus. This antigen can be added to the culture expansion media to increase efficacy, antigen-presenting potential and cytotoxicity of expanded gamma delta T cells. Suitably, antigens may be provided in various formats, including but not limited to, immobilised antigens or antibodies, irradiated tumour cell lines, artificial antigen presenting cells and addition of synthetic soluble antigens. The antigen may be added to the culture expansion media on the first day of culturing. In embodiments the virus can be selected from influenza, HIV, Hepatitis C, Hepatitis B, Herpes variants, Cytomegalovirus (CMV), Epstein Barr Virus, Chickenpox, Papillomavirus, Ebola, Varicella Zoster virus or Smallpox. Alternatively the antigen can be an antigen found in a cell infection, bacterial infection, fungal infection or protozoan infection. In particular the target antigen can be from influenza, HIV, Hepatitis C, Hepatitis B, Herpes variants, Cytomegalovirus (CMV), Ebola virus, Epstein Barr Virus, Chickenpox, Papillomavirus, Varicella Zoster virus or Smallpox.

Suitably, the antigen may include an active or inactivated viral fragment, peptide, a protein, antigenic segment or the like from such a virus organism.

Suitably, the antigen may include a tumour-specific antigen which is present only on tumour cells and not on any other cells and/or a tumour-associated antigen which is present on some tumour cells and also some normal cells. Such tumour-specific antigens may include, but are not limited to, carcinoembryonic antigen, CA-125, MUC-1, epithelial tumour antigen and a MAGE-type antigen including MAGEA1, MAGEA3, MAGEA4, MAGEA12, MAGEC2, BAGE, GAGE, XAGE1B, CTAG2, CTAG1, SSX2, or LAGE1 or combinations thereof.

Suitably, a lysate of an infected cell, a necrotic cell, or a cancer cell may be utilised to provide a suitable antigen. In embodiments the antigen may be a synthesised antigen, for example, a synthetic peptide. Alternatively, the antigen may be harvested from a subject. Suitably, around 0.02-2 micro grams per ml of antigen may be provided to the cells during the culturing step.

In embodiments, factors which encourage proliferation of gamma delta T cells and maintenance of cellular phenotype such as IL-2, IL-15 or IL-18 (Garcia V. et al., 1998, Nussbaumer O. et al., 2013) may be provided in the step of culturing the blood mononuclear cells. Suitably, in such embodiments IL-2, IL-15 or IL-18 or combinations thereof may be provided in the range of 50-2000 U/ml, more preferably 400-1000 U/ml to the culturing medium. Culture is typically performed at 34 to 38 deg. C., more preferably 37 deg. C. in the presence of 2 to 10%, more preferably 5% CO₂. Culture medium may be added depending on the number of cultured cells. Suitably serum may be added in an amount of 0.1 to 20% to the culture solution. As the serum, fetal calf serum AB serum, or auto-plasma may be used, for example.

In embodiments, factors which encourage the revival of exhausted or anergic gamma delta T cells may be added to the culture medium. Suitably, these factors may include cytokines such as IL-15 or IL-18 or antibodies targeting specific immune check-point inhibitor receptors or ligands for example anti-PD-L1 antibody (Chang K. et al., 2014) but may also include antibodies directed to CTLA-4, PD-1, PD-2, LAG3, CD80, CD86, B7-H3, B7-H4, HVEM, BTLA, KIR, TI M3 or A2aR.

In embodiments, the providing step may include the collection of blood or umbilical cord blood from a donor subject. Such blood collection may be of about 15 to 25 ml of blood. In embodiments the providing step may include a collecting step wherein the step of collecting is the collection of at least gamma delta T cells from the first subject in a single collection process. In embodiments the collecting step can be over multiple collection sessions.

In an embodiment of the invention the process for providing gamma delta T cells can comprise an analysing step of determining at least one characteristic of a cell collected from a first subject. In embodiments at least one characteristic of a cell can be a DNA or RNA sequence or amino acid sequence of the cell, a proteome of the cell or a cell surface marker of the cell. In embodiments the process can include a step of tissue typing the gamma delta T cells. Gamma delta cell surface marker characteristics may include (but are not limited to) CD3, CD4, CD8, CD69, CD56, CD27 CD45RA, CD45, TCR-Vg9, TCR-Vd2, TCR-Vd1, TCR-Vd3, TCR-pan g/d,NKG2D, monoclonal chemokine receptor antibodies CCR5, CCR7, CXCR3 or CXCR5 or combinations thereof. This typing may include genotypic or phenotypic information. Phenotypic information may include observable or measurable characteristics at the microscopic, cellular, or molecular level. Genotypic information may relate to specific genetic variations or mutations, for example, of the human leukocyte antigen (HLA type of the donor). Suitably the gamma delta T cells may provide banks of clinical grade cell lines that can be expanded and differentiated for use in a large number of patients. In embodiments, gamma delta T cells may be expanded ex vivo from umbilical cord blood starting material and combined from multiple donors to generate sufficient numbers of gamma delta T cells to populate a cell bank. In embodiments such a bank would suitably be populated with gamma delta T cells obtained from healthy volunteer donors of blood group O that are selected to maximize the opportunity for Human Leukocyte Antigens (HLA) matching and thereby minimise the risk of allograft rejection or need for substantial use of immunosuppressive drugs. For instance such banks for UK/EU patients may comprise the following which would allow treatment of a significant percentage of the UK/EU population with reduced risk of rejection:

| HLA-A | HLA-B | HLA-DR |
|---|---|---|
| A1 | B8 | DR17(3) |
| A2 | B44(12) | DR4 |
| A3 | B7 | DR15(2) |
| A2 | B7 | DR15(2) |
| A2 | B44(12) | DR7 |
| A2 | B62(15) | DR4 |
| A1 | B57(17) | DR7 |
| A3 | B35 | DR1 |
| A29(19) | B44(12) | DR7 |
| A2 | B60(40) | DR4 |
| A2 | B8 | DR17(3) |
| A2 | B27 | DR1 |
| A2 | B44(12) | DR13(6) |
| A3 | B7 | DR4 |
| A1 | B8 | DR4 |
| A2 | B57(17) | DR7 |
| A2 | B60(40) | DR13(6) |
| A11 | B35 | DR4 |
| A2 | B44(12) | DR11(5) |
| A24(9) | B7 | DR15(2) |
| A30(19) | B13 | DR7 |
| A31(19) | B60(40) | DR4 |
| A3 | B7 | DR1 |
| A11 | B35 | DR1 |
| A3 | B65(14) | DR13(6) |

In embodiments collected and processed gamma delta T cells can be banked for future use at a cell bank or depository. Accordingly, the cells may be stored in a cryoprotectant such as DMSO or CryoStor™ and subjected to a controlled rate of freezing and storage with in liquid nitrogen. The gamma delta T cells may be stored in a unitised storage of defined units or dosages as required for a single or multiple treatment steps.

In an embodiment the process can comprise a step of treating a population of cells collected from a first subject with an agent to enhance the storage, viability or therapeutic ability of gamma delta T cells within the collected sample. In an embodiment, the process can include a preserving step wherein a cryopreservation agent is provided to gamma delta T cells in the sample of gamma delta T cells.

In embodiments a gamma delta T cell can be a phosphoantigen isopentenyl pyrophosphate (IPP) expanded human Vγ9Vδ2 T cell.

In embodiments a gamma delta T cell can be an expanded human Vδ1 T or Vδ3 T cell.

According to a second aspect of the invention there is provided a method of treating an infection or cancer in an individual comprising the step of providing said individual with gamma delta T cells obtained from a different individual. Thus, donor gamma delta T cells are used for the treatment of an infection, for example, of a virus, fungi or protozoa, or for treatment of a cancer in a recipient subject wherein the donor and the recipient are not the same individual.

The method of administration to provide the gamma delta T cells to the recipient subject may include intravenous, intradermal, or subcutaneous injection. Administration may be into an affected area or systemically to the individual.

In embodiments there is provided gamma delta T cells from a first subject for use in the treatment of a second different subject infected by a virus, fungi or protozoa wherein said treatment of the subject is allogeneic.

In embodiments there is provided gamma delta T cells from a first subject for the treatment of a second different subject infected by virus, wherein said virus is selected from HIV, influenza, or hepatitis, wherein said treatment is allogeneic. In an embodiment the virus can be hepatitis B or hepatitis C, influenza, Herpes variants, Cytomegalovirus (CMV), Epstein Barr Virus, Chickenpox, Papillomavirus, Varicella Zoster virus or Smallpox.

In embodiments the influenza virus can be influenza A (Flu A) virus. In embodiments the influenza virus can be an avian or swine—origin pandemic influenza virus, for example, H5N1, H7N3, H7N7, H7N9 and H9N2 (avian subtypes) or H1N1, H1N2, H2N1, H3N1, H3N2 H2N3 (swine subtypes).

In embodiments there is provided gamma delta T cells for the treatment of a subject with cancer wherein said treatment is allogeneic.

In embodiments there is provided gamma delta T cells from a first subject for use in the treatment of a second subject wherein the second subject is suffering from at least one of a viral, fungal or protozoan infection. In embodiments the subject being provided with gamma delta T cells can be simultaneously, sequentially or separately administered with immunosuppressive drugs. The administration of immunosuppressive drugs can help mitigate any detrimental immune system response to the gamma delta T cells.

In embodiments, there is provided gamma delta T cells for the treatment of a subject with Epstein-Barr virus-induced lymphoproliferative disease (EBV-LPD).

Epstein-Barr virus (EBV) is a member of the gamma herpes virus family and is prevalent in Western populations (>90% of adults are seropositive). EBV is maintained as a latent infection by the host's cytotoxic T cells (CTLs) which prevent viral reactivation thus allowing EBV to persist asymptomatically as a latent infection in host B cells.

EBV is associated with a number of malignancies of B cell origin such as Burkitt's lymphoma (BL), Hodgkin's disease (HD) and post-transplant lymphoproliferative disease (PTLD) in addition to cancers of epithelial origin such as nasopharyngeal carcinoma (NPC) and gastric cancer.

PTLD is a common risk associated with solid organ transplantation and hematopoietic stem cell transplantation.

In embodiments there is provided gamma delta T cells from a first subject for use in the treatment of a second subject with an EBV-associated malignancy.

In embodiments there is provided gamma delta T cells of one or more specific gamma delta TCR isotypes for the treatment of different viral indications. For example, Vδ2^{pos} subtypes may be most efficacious in the treatment of HIV and influenza infection (Wallace M. et al., 1996, Tu W. et al. 2011), whilst evidence exists for the role of at least two gamma delta T cell subtypes in the control of EBV infected cells; Vδ1^{pos} (Farnault L, et al., 2013) and Vδ2^{pos} cells (Xiang Z. et al., 2014). Suitably, combinations of gamma delta T cell subtypes may be chosen and administered to the patient to increase the effectiveness of the gamma delta T cell therapy. Suitably, these may comprise single isotype gamma delta T cell populations generated using discrete culturing conditions or a multivalent gamma delta T cell population generated concomitantly using a defined single set of cell culture parameters.

The gamma delta T cells used in the second aspect of the present invention may be any of those described in the first aspect of the present invention, i.e. after the steps of providing and culturing as discussed above.

In a third aspect of the present invention, there is provided a process for providing gamma delta T cells autologously to a subject comprising the steps
providing a sample of gamma delta T cells from a subject;
culturing the gamma delta T cells to allow them to be administered back to the subject.

Any of the steps of providing and culturing described above for the first aspect of the present invention may be applied to the third aspect of the present invention. For example, the step of culturing the gamma delta T cells may include steps for changing the gamma delta T cell surface receptor profile, as discussed above.

In a fourth aspect of the present invention there is provided a method of treating an infection or cancer in an individual comprising the step of providing said individual with gamma delta T cells obtained from that individual, wherein the gamma delta T cells have been provided by a process as described in the third aspect of the present invention.

In embodiments the cancer can be a myeloma or melanoma. In embodiments a cancer can include but is not limited to a tumour type, including gastric cancer, renal cell carcinoma, hepatocellular carcinoma, pancreatic cancer, acute myeloid leukaemia, multiple myeloma, acute lymphoblastic leukaemia, non-small cell lung cancer, EBV-LPD, Burkitt's lymphoma and Hodgkin's disease.

According to a further aspect of the present invention there is provided a pharmaceutical composition comprising a gamma delta T cell of any of the processes of the present invention.

In embodiments the composition comprises a unified dose of gamma delta T cells suitable to provide to an individual to provide a therapeutic effect.

In embodiments the pharmaceutical composition can include a total dose of over 25×10⁹ gamma delta T cells per person.

In embodiments, there is provided a pharmaceutical composition comprising gamma delta T cells and an antibody immunotherapy for use in the treatment of cancer.

In embodiments an antibody immunotherapy can be an immune cascade blocking agent such as PD-1, PDL-1 and/or CTLA-4 inhibitor, PD-1, PDL-1 and

CTLA-4 inhibitors, for example, as being developed by Roche and Bristol Myers Squibb.

In embodiments the pharmaceutical composition can include an antibody capable of blocking CTLA-4 inhibitory signals. Blocking of CTLA-4 signals allow

T lymphocytes to recognise and destroy cells. In embodiments such an antibody can be Ipilimumab (MDX-010, MDX-101).

In embodiments the antibody can inhibit Programmed death-ligand 1 (PDL-1). In embodiments such an antibody can be selected from MPDL3280A (Roche) or

MDX-1105.

In embodiments the pharmaceutical composition may be combined with a cytokine, for example, IL-2 or IL-12. In embodiments the pharmaceutical composition may include interferon gamma.

In embodiments, there is provided a pharmaceutical composition comprising gamma delta T cells and a chemotherapeutic for use in the treatment of cancer.

In embodiments, there is provided a pharmaceutical composition comprising gamma delta T cells and a therapeutic for use in the treatment of virus.

In embodiments the pharmaceutical composition can be used as a therapeutic or a prophylactic agent for cancer or infections.

In embodiments of the invention, the gamma delta T cell can be a Vγ9Vδ2 T cell.

Preferred features and embodiments of each aspect of the invention are as for each of the other aspects mutatis mutandis unless context demands otherwise.

Each document, reference, patent application or patent cited in this text is expressly incorporated herein in their entirety by reference, which means it should be read and considered by the reader as part of this text. That the document, reference, patent application or patent cited in the text is not repeated in this text is merely for reasons of conciseness.

Reference to cited material or information contained in the text should not be understood as a concession that the material or information was part of the common general knowledge or was known in any country.

Throughout the specification, unless the context demands otherwise, the terms ‘comprise’ or ‘include’, or variations such as ‘comprises’ or ‘comprising’, ‘includes’ or ‘including’ will be understood to imply the includes of a stated integer or group of integers, but not the exclusion of any other integer or group of integers.

Embodiments of the invention will now be described by way of example only with reference to the accompanying figures in which

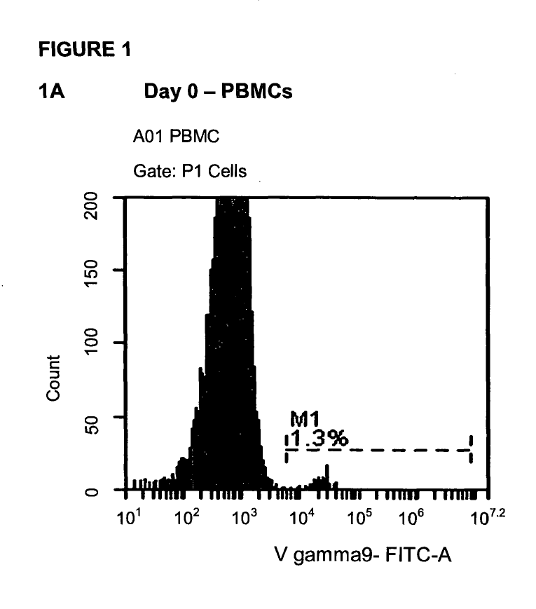

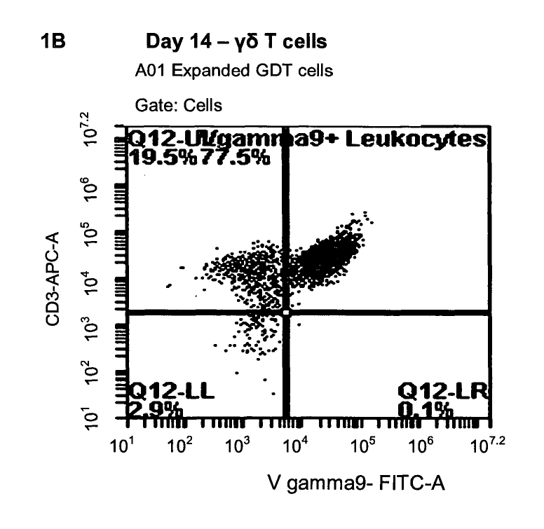

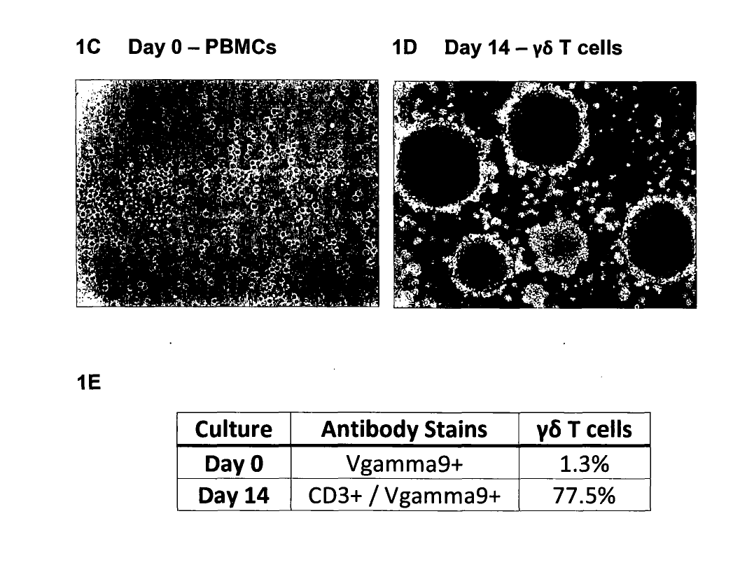

FIG. 1 illustrates immunophenotyping of starter culture PBMCs and following 14 days of expansion in culture to selectively activate and proliferate the γδ T cell population (Vgamma9 Vdelta2) wherein flow cytometry immunophenotyping of cell populations is used at the start of the culturing process (day 0), using PBMCs isolated from human blood as the starting material and at the end of the selective expansion process (day 14):—A—histogram of isolated PBMCs on day 0 stained with anti-Vgamma9-FITC antibody to detect the percentage of γδ T cells in starting population of PBMCs (1.3% of PBMCs are δδ T cells): B—Dot plot analysis of the cell population after 14 days of selective culturing stained with anti-CD3 (T cells) and anti-Vgamma9 (γδ T cells (77.5% of T cells are γδ T cells): C,D—bright field images of isolated PBMCs (C) and cell population after 14 days of expansion in culture (D): E—Table indicating the percentages of γδ T cells present within each cell culture population;

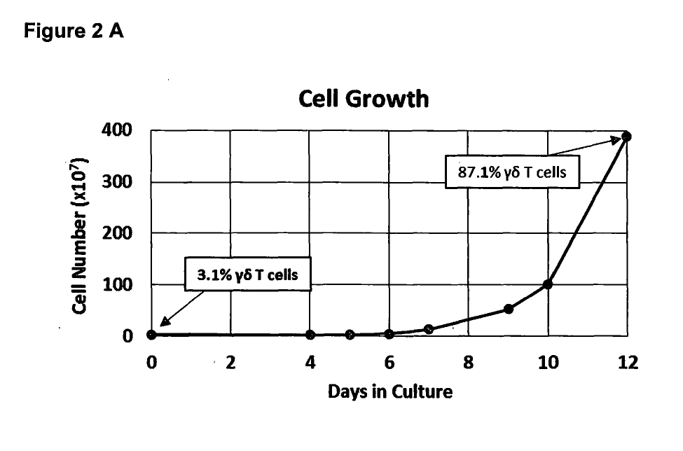

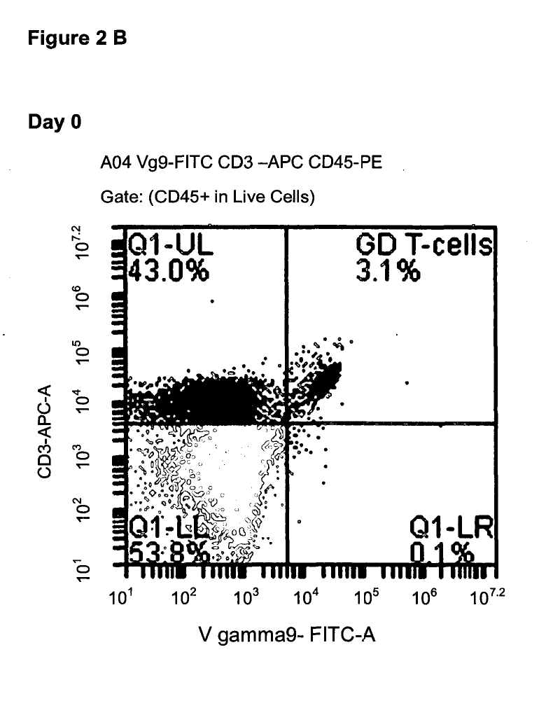

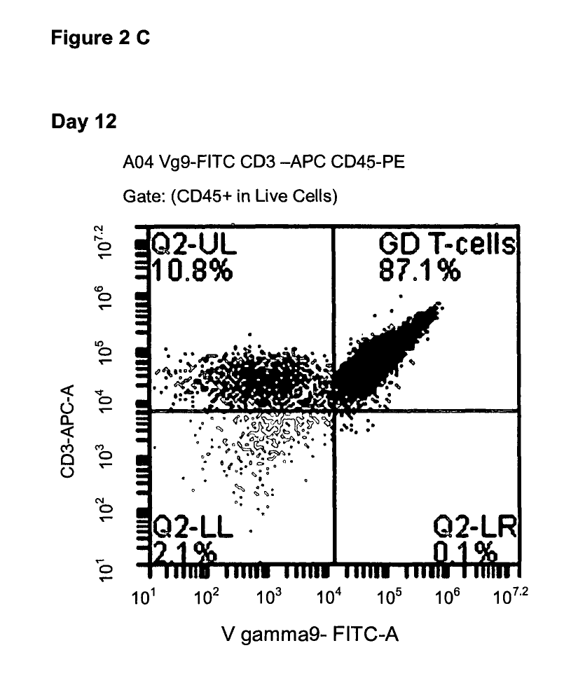

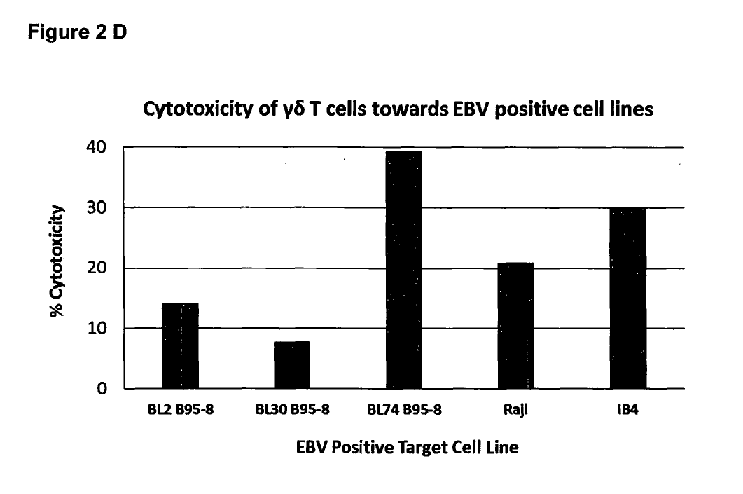

FIG. 2 illustrates the exponential growth of cells selectively expanded in culture to activate and proliferate the γδ T cell population (Vgamma9 Vdelta2) wherein significant numbers of high purity γδ T cells are generated by day 12 which are demonstrated to be potent effectors of cancer cell cytolysis using a panel of EBV-positive lymphoma cell lines in vitro—Flow cytometry immunophenotyping of cell populations is used at the start of the culturing process (day 0), using PBMCs isolated from human blood as the starting material and later in the selective expansion process (day 12):—A—Growth chart indicating the total number of viable cells in culture throughout the first 12 days of expansion with a total of 4×10⁹ cells achieved by day 12: B,C—Flow cytometry analysis of starting PBMCs (B) and the cell population following 12 days of selective expansion in culture (C) demonstrating 3.1% (day 0) and 87.1% (day 12) γδ T cells (anti-Vgamma9) respectively: D—γδ T cells were incubated with five EBV positive target cells lines (BL2 B95-8, BL30 B95-8, BL74 B95-8, Raji and 1B4) at an effector:target cell ratio of 5:1 for 16 hours—γδ T cell elicited cytolysis was measured using the non-radioactive Cytotox96 assay and is expressed as a percentage of maximum target cell lysis; and

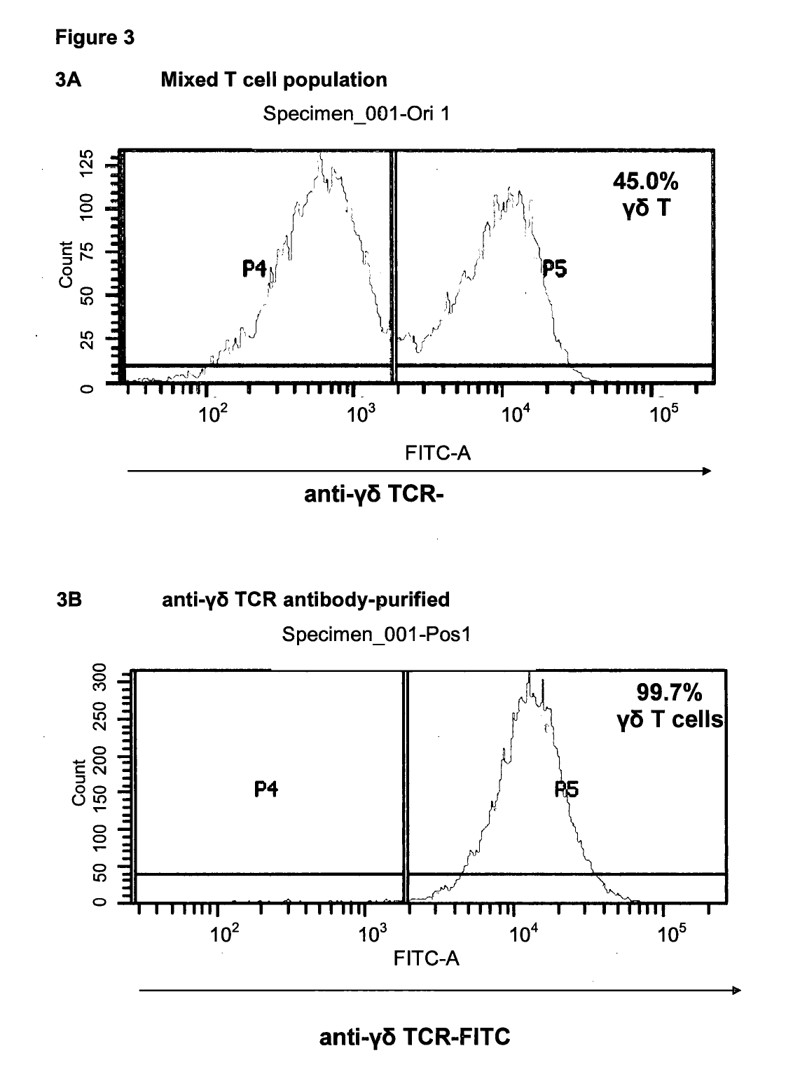

FIG. 3 illustrates an antibody-mediated purification method employed to isolate discrete cellular phenotypes from a heterogeneous cell population wherein in this example, cells have been selected with a pan-anti-γδ T cell receptor antibody to obtain a γδ T cell population in extremely high purity—Flow cytometry immunophenotyping analysis of the cell population prior to purification (A) and following purification (B) using an anti-γδ T cell receptor-FITC conjugated antibody demonstrates that γδ T cells are obtained at 99.7% purity from a 45% γδ T cell starting material.

Gamma Delta T cells may be culture expanded using the technique outlined by Nicol A. J. et. al., 2011 Peripheral blood mononuclear cells (PBMCs) were isolated by density gradient centrifugation using Ficoll-Paque (GE Healthcare, Buckinghamshire, UK) and Vγ9Vδ2 T cells selectively proliferated by culture of

PBMCs in RPMI 1640 media (Lonza, Walkersville, Md., USA) supplemented with 10% human AB plasma (Lonza), L-glutamine (2 mM; Lonza) and gentamycin (40 μg; Pfizer, Bentley, WA, Australia). Recombinant human IL-2 (700 IU ml-1; Novartis, Basel, Switzerland) and zoledronate (1 μM; Novartis) were added on day 0 and additional IL-2 (350 IU ml-1) was added every 2-3 days during the culture period. After 7-14 days culture, purified effector cell populations containing 70-95% Vγ9Vδ2 T cells were obtained for in vitro functional assessment by depletion of CD4+, CD8+ and CD56+ cells using miniMACS (Miltenyi Biotec, Bergisch Gladbach, Germany).

The autologous treatment of patients with solid tumours with ex vivo expanded Vγ9Vδ2 T cells has been demonstrated to provide clinical benefit (Noguchi et al., 2011). Additionally, allogeneic treatment with HLA-matched, ex vivo expanded αβ TCR-positive cytotoxic T lymphocytes (CTLs) has proven to be efficacious in the treatment of EBV-PTLD (Hague T et al., 2007). The present inventors consider therefore that the treatment of cancer and viral infections with allogeneic gamma delta T cells is both feasible and likely to provide demonstrable therapeutic benefit to the patient.

Although the invention has been particularly shown and described with reference to particular examples, it will be understood by those skilled in the art that various changes in the form and details may be made therein without departing from the scope of the present invention.

### REFERENCES

Chan D., Gibson H. M., Aufiero B. M., Wilson A. J., Hafner M.S., Mi Q. S. and Wong H. K. Differential CTLA-4 expression in human CD4+ versus CD8+ T cells is associated with increased NFAT1 and inhibition of CD4+ proliferation. Genes Immun., 2014, 15(1):25-32.
Chang K, Svabek C, Vazquez-Guillamet C, Sato B, Rasche D, Wilson S, Robbins P, Ulbrandt N, Suzich J, Green J, Patera A C, Blair W, Krishnan S, Hotchkiss R. Targeting the programmed cell death 1: programmed cell death ligand 1 pathway reverses T cell exhaustion in patients with sepsis. Crit Care., 2014, 8:R3
Farnault L, Gertner-Dardenne J., Gondois-Rey F., Michel G., Chambost H., Hirsch I and Olive D. Clinical evidence implicating gamma-delta T cells in EBV control following cord blood transplantation. Bone Marrow Transplant., 2013, 11:1478-9.
Garcia V. E., Jullien D., Song M., Uyemura K., Shuai K., Morita C. T. and Modlin R. L. IL-15 Enhances the Response of Human γδ T Cells to Nonpetide Microbial Antigens J. Immunol., 1998, 160: 4322-4329
Hague T, Wilkie G M, Jones M M, Higgins C D, Urquhart G, Wingate P, Burns D, McAulay K, Turner M, Bellamy C, Amlot P L, Kelly D, MacGilchrist A, Gandhi M K, Swerdlow A J and Crawford D H. Allogeneic cytotoxic T-cell therapy for EBV-positive posttransplantation lymphoproliferative disease: results of a phase 2 multicenter clinical trial. Blood. 2007 Aug 15,110(4):1123-31.
Iwasaki M., Tanaka Y., Kobayashi H., Murata-Hirai K., Miyabe H., Sugie T., Toi M. and Minato N. Expression and function of PD-1 in human cd T cells that recognize phosphoantigens. Eur. J. Immunol., 2011, 41: 345-355.
Meraviglia S, Eberl M, Vermijlen D, Todaro M, Buccheri S, Cicero G, La Mendola C, Guggino G, D'Asaro M, Orlando V, Scarpa F, Roberts A, Caccamo N, Stassi G, Dieli F, Hayday AC. In vivo manipulation of Vgamma9Vdelta2 T cells with zoledronate and low-dose interleukin-2 for immunotherapy of advanced breast cancer patients. Clin Exp Immunol., 2010, 161(2):290-7.
Nicol A. J., Tokuyama H., Mattarollo S. R., Hagi T., Suzuki K., Yokokawa K. and Nieda M. Clinical evaluation of autologous gamma delta T cell-based immunotherapy for metastatic solid tumours. Br J Cancer, 2011, 105(6):778-86.
Noguchi A, Kaneko T, Kamigaki T, Fujimoto K, Ozawa M, Saito M, Ariyoshi N and Goto S. Zoledronate-activated Vγ9γδ T cell-based immunotherapy is feasible and restores the impairment of γδ T cells in patients with solid tumors. Cytotherapy. 2011 Jan;13(1):92-7.
Nussbaumer O., Gruenbacher G., Gander H., Komuczki J., Rahm A. and Thurnher M. Essential requirements of zoledronate-induced cytokine and γδ T cell proliferative responses. J. Immunol., 2013, 191(3):1346-55.
Tanaka Y, Morita C T, Tanaka Y, Nieves E, Brenner M B, Bloom B R. Natural and synthetic non-peptide antigens recognized by human gamma delta T cells. Nature. 1995, 375(6527):155-8.
Thompson K., Roelofs A. J., Jauhiainen M., Monkkonen H., Monkkonen J. and Rogers M. J. Activation of γδ T Cells by Bisphosphonates (Chapter from Osteoimmunoligy, Advances in Experimental Medicine and Biology 658, DOI 10.1007/978-1-4419-1050-9_2)
Tu W., Zheng J., Liu Y., Sia S. F., Liu M., Qin G., Ng I. H., Xiang Z., Lam K. T., Peiris J. S. and Lau Y. L. The aminobisphosphonate pamidronate controls influenza pathogenesis by expanding a gammadelta T cell population in humanized mice. J Exp Med., 2011, 208(7):1511-22.
Wallace M., Bartz S. R., Chang W. L., Mackenzie D. A., Pauza C. D. and Malkovsky M. Gamma delta T lymphocyte responses to HIV. Clin Exp Immunol., 1996, 103(2):177-84.
Wang H., Sarikonda G., Puan K., Tanaka Y., Feng J., Giner J., Cao R., Monkkonen J., Oldfield E. and Morita C.T. Indirect Stimulation of Human Vg2Vd2 T Cells through Alterations in Isoprenoid Metabolism. J. Immunol., 2011, 187: 5099-113.
Xiang Z., Liu Y., Zheng J., Liu M., Lv A., Gao Y., Hu H., Lam K. T., Chan G. C., Yang Y., Chen H., Tsao G. S., Bonneville M., Lau Y. L. and Tu W. Targeted activation of human Vγ9Vδ2-T cells controls epstein-barr virus-induced B cell lymphoproliferative disease. Cancer Cell, 2014, 26(4):565-76.

1. A process to provide gamma delta T cells from a first subject to a second allogeneic subject wherein the process comprises:
providing a sample comprising gamma delta T cells from a first subject; and
culturing the gamma delta T cells present in the sample to allow the gamma delta T cells to be administered to a second subject.

2. A process as claimed in claim 1 wherein the step of culturing the gamma delta T cell provides for an increase in the number of gamma delta T cells in the sample relative to other cell types in the sample.

3. A process as claimed in claim 1 wherein the culturing step comprises a step of purifying gamma delta T cells in the sample from other cell types in the sample.

4. A process as claimed in claim 3 wherein the step of purifying uses an anti-gamma delta T cell antibody to purify and isolate the gamma delta T cells from the other cell types in the sample.

5. A process as claimed in claim 1 wherein the gamma delta T cells are cultured or purified to provide more than 10% of the total number of cells present in the sample.

6. A process as claimed in claim 1 wherein the culturing step comprises one or more sub-steps to reduce or eliminate one or more gamma delta T cell surface receptor types present on the gamma delta T cells in the sample from the first subject.

7. A process as claimed in claim 1 wherein the culturing step includes a step that induces the expression, in the gamma delta T cells, of a surface receptor type(s) that was not present on the surface of the uncultured gamma delta T cells from the first subject or that induce an increase in the amount of expression of a cell surface receptor type(s) that was present on the surface of the gamma delta T cells from the first subject.

8. A process as claimed in claim 1 that further comprises a step of monitoring the cell surface receptor profile of the gamma delta T cells in the sample.

9. A method of treating an infection or cancer in an individual comprising the step of providing said individual with gamma delta T cells obtained from a different allogeneic individual.

10. A method as claimed by claim 9 wherein the gamma delta T cells are provided by a process comprising providing a sample comprising gamma delta T cells from the different allogenic individual and culturing the gamma delta T cells present in the sample to allow the gamma delta T cells to be administered to the individual receiving the treatment for infection or cancer.

11. (canceled)

12. A method of treating as claimed in claim 9 wherein said individual is a first subject and the gamma delta T cells are obtained from a second subject that is a different allogenic individual to the first subject, the method comprising administering gamma delta T cells from the second subject to the first subject, simultaneously, separately or sequentially with an immunosuppressive drug.

13. The method of treating of claim 9 wherein the infection is at least one of a viral, fungal or protozoan infection.

14. (canceled)

15. (canceled)

16. A method of treating cancer or an infection as claimed in claim 9 wherein the infection is selected from a virus, fungi or protozoa infection comprising administering to an individual in need of such treatment gamma delta T cells and an antibody immunotherapy.

17. The method according to claim 16 wherein the gamma delta T cells and the antibody immunotherapy are administered simultaneously.

18. The method according to claim 16 wherein the gamma delta T cells and the antibody immunotherapy are administered separately.

19. The method according to claim 16 wherein the gamma delta T cells and the antibody immunotherapy are administered sequentially.

20. A pharmaceutical composition comprising gamma delta T cells and an immunosuppressive drug.

21. The method according to claim 16 wherein the method is a method of treating a virus, fungi or protozoa infection and the step of administering to an individual in need of such treatment comprises administering gamma delta T cells to a first individual in need of such treatment, the gamma delta T cells having been obtained from a second individual that is allogeneic with respect to the first individual.

22. The method according to claim 16 wherein the step of administering to an individual in need of such treatment comprises administering gamma delta T cells to a first individual in need of such treatment, the gamma delta T cells having been obtained from a second individual that is allogeneic with respect to the first individual.

23. a method of treating an infection or cancer in an individual as claimed in claim 9 wherein said individual is provided with sufficient numbers of gamma delta t cells to exert a therapeutic effect to said individual wherein the gamma delta t cells are provided from a different allogeneic individual.
